(19) United States
(12) Patent Application Publication
(10) Pub. No.: US 2006/0178604 A1
(43) Pub. Date: Aug. 10, 2006

(54) Blood pumping system

(76) Inventor: Robert J. Alderman, Canyon Lake, TX (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/052,582

(22) Filed: Feb. 7, 2005

(51) Int. Cl. A61H 9/00; A61N 1/362

(52) U.S. Cl. 601/149; 600/16; 601/151

(57) ABSTRACT

The human heart is assisted in moving blood through blood vessels by alternately applying and relieving pressure about a blood vessel with a vessel constrictor. The blood flow is restricted to one direction along the blood vessel by restrictors. The application of pressure to the blood vessel may be timed with the heart beat.

### TECHNICAL FIELD

The present disclosure is generally related to blood pumping systems and, more particularly, is related to a system and method for pumping blood through the body of a patient.

### BACKGROUND OF THE INVENTION

There exists a shortage in donor hearts for heart transplant operations. The shortage of donor hearts is so severe that some patients, who are on heart transplant waiting list, die from heart failure before a donor heart can be found. Some of the problems associated with human-to-human heart transplant include the shortage of donor hearts; the need to match tissue type so that the donor heart is accepted by the body of the heart transplant patient; inability to anticipate when a donor heart will become available and schedule procedures in advance; and the need for geographical proximity. Once a donor heart has been harvested, the donor heart is refrigerated until it is transplanted in the body of a patient. However, even though the donor heart is refrigerated, it will degrade over time. Consequently, a patient needs to be geographically proximal to where the donor heart is transplanted so as to reduce the time that the donor heart is refrigerated.

Due in part to the problems associated with human-to-human heart transplants, attempts have been made to provide heart transplant patients with “artificial” hearts and animal hearts. Such attempts have generally been experimental and have produced unsatisfactory results. In non-human-to-human heart transplant experiments, a patient has their natural heart removed and replaced by a non-human (artificial or animal) heart. The patient is then dependent upon the transplanted non-human heart until a human donor heart becomes available.

An advantage of a artificial (man-made) heart is that the heart can be stored at a hospital waiting for a patient, thereby negating the need for geographical proximity, and procedures can be scheduled well in advance. Furthermore, an artificial heart can be made from materials that will not be rejected by the patient's body. However to date, artificial hearts have not proved effective. One reason, among others, that artificial hearts have been ineffective is that they can cause blood clotting, which can lead to strokes and other medical complications. To reduce the risk of blood clotting, patients generally receive drugs that thin their blood. However, blood-thinning drugs also have medical risks associated with them.

Animal-to-human heart transplants have some of the general advantages of artificial hearts such as animal hearts can be easily procured and animal-to-human transplants can be scheduled in advance. However, animal-to-human heart transplants have so far proved unsuccessful for at least the reason that the human body normally rejects the foreign animal tissue.

Thus, a heretofore-unaddressed need exists in the industry to address the aforementioned deficiencies and inadequacies. Among other needs, there exists the need for an apparatus that can assist a patient's heart in pumping blood so that the patient can survive until a human donor heart becomes available. There also exists the need for an apparatus that can pump blood through the patients body even when the patient's heart has failed or been removed.

### SUMMARY OF THE INVENTION

Briefly described, the present invention comprises a method of assisting the heart of a human body to move blood through the blood vessels of the body by alternately applying pressure to and relieving pressure from the exterior of a blood vessel of the body. The flow of blood through the vessel is restricted to one direction.

One embodiment of the method includes coordinating the application of pressure to the blood vessel with the heart beat of the body.

A system for pumping blood through the body includes a blood restrictor that allows blood flow though the blood vessel in a first direction and inhibits blood flow in the opposite direction and a blood vessel constrictor that constricts about the blood vessel to cause blood within the blood vessel to flow through the blood vessel and its blood restrictor.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a diagram of a patient and blood pumping system.

FIG. 2 is a diagram of the circulatory system of the patient of FIG. 1.

FIG. 3 is a top view of a blood vessel constrictor.

FIG. 4 is a cross sectional side view of the blood vessel constrictor of FIG. 3.

FIGS. 5A-5C are partial views of the blood vessel constrictor in operable states wrapped around the leg of the patient.

FIG. 6 is a diagram of another embodiment of a blood pumping system.

FIG. 7 is a diagram of an internal blood pumping system.

### DETAILED DESCRIPTION

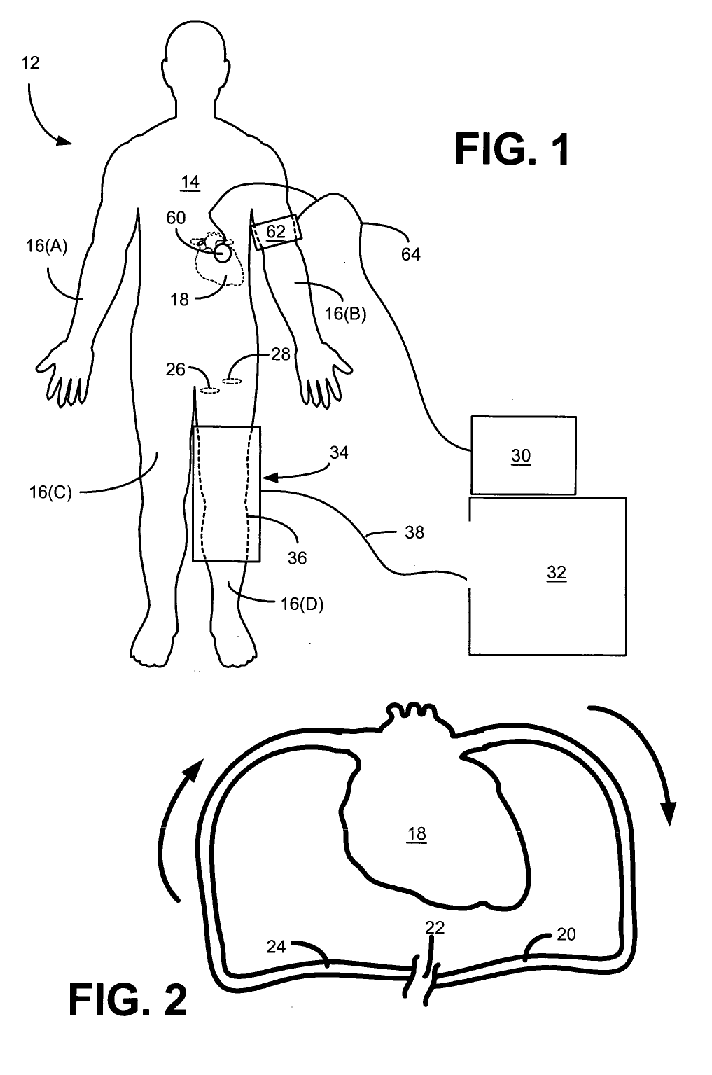

FIG. 1 illustrates an embodiment of a blood pump system 10 for pumping and/or enhancing the pumping of blood through patient 12. In some embodiments, the blood pumping system 10 replaces the patient's heart such that the blood pumping system 10 is solely responsible for pumping blood through the patient's body. In other embodiments, the blood pumping system 10 works in conjunction with the patient's heart to assist the heart in pumping blood through the patient's body. In this description, embodiments are described in which the blood pumping system 10 assists a heart in its pumping. However, those are non-limiting embodiments and are provided for the sake of clarity.

For the purposes of illustration, the patient 12 is depicted as a human having a torso 14, limbs 16, which include arms 16(A) and 16(B) and legs 16(C) and 16(D), and a blood circulatory system comprising a heart 18, arteries 20, capillaries 22, and veins 24. (See FIG. 2.) The natural blood flow direction of the circulatory system is from the heart 18 through the arteries 20 to the capillaries 22 and through the capillaries 22 to the veins 24 and back to the heart 26. The human body and its circulatory system are well known and are not discussed in detail.

In one embodiment, a blood pumping system 10 includes an arterial blood restrictor 26 and a venial blood restrictor 28, which are implanted in an artery 20 and a vein 24, respectively. In this embodiment, the blood restrictors 26 and 28 are one-way check valves that inhibit blood flow counter to the natural blood flow of the body. In other words, the arterial blood restrictor 26 inhibits the back flow of blood towards the heart; and, the venial blood restrictor 28 inhibits the back flow of blood away from the heart. In this embodiment, the arterial blood flow restrictor 26 and the venial blood flow restrictor 28 are disposed in the body proximal to the torso 14 such that a significant amount of blood within the leg 16(C) is bounded by the blood restrictors 26 and 28. Exemplary blood restrictors include both mechanical valves such as, but not limited to, one-way check valves and biological valves such as, but not limited to, pig valves. Typically, blood restrictors are similar to heart replacement valves, which are well known in the art.

The blood pumping system 10 also includes a controller 30, an actuator 32 and a blood vessel constrictor 34. As will be explained in detail hereinbelow, the blood vessel constrictor 34 circumscribes and extends externally along a therapy portion 36 of the leg 16(C). The blood vessel constrictor 34 is coupled to the actuator 32 by a pressure line 36 which carries a fluid such as air, gas, liquid such as, but not limited to, saline liquid and silicone liquid from the actuator 32 to the blood vessel constrictor 34. Responsive to activation by the actuator 32, the blood vessel constrictor 34 inflates and deflates such that pressure and/or suction are applied to the therapy portion 36 of the leg 16(C). In some embodiments, the blood vessel constrictor 34 is cuff like and is similar to a cuff of the type used to measure blood pressure or the like.

In one embodiment, the patient 12 is coupled to a heart monitor 60 and a blood pressure monitor 62, which are coupled to the controller 30 by an electrical connector 64. The controller 30 monitors the patient's blood pressure and heart rate and causes the actuator 32 to inflate and deflate the blood vessel constrictor 34. In some preferred embodiments, the controller 30 maintains the patient's blood pressure within a desired range using the blood vessel constrictor 34. In addition, in some embodiments, the controller 30 causes the blood vessel constrictor 34 to inflate and deflate in rhythm with the patient's heart rate. The actuation of the blood vessel constrictor 34 can be offset from the pumping of the heart such that the blood vessel constrictor 34 is generally in rhythm with the systolic blood flow through the leg 16.

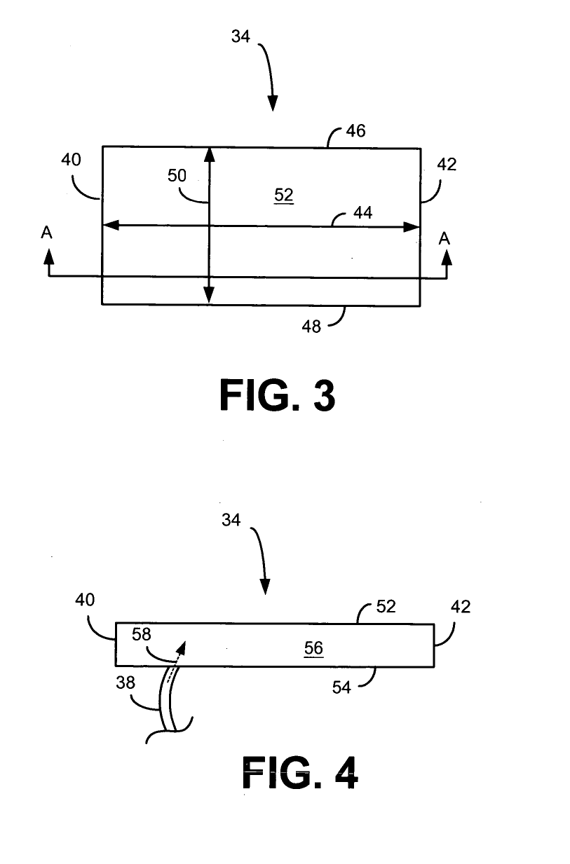

FIG. 3 illustrates the blood vessel constrictor 34 when the blood vessel constrictor 34 is not disposed around the leg 16(C). The blood vessel constrictor 34 has opposed ends 40 and 42, which define a longitudinal length 44, and opposed ends, 46 and 48, which define a transverse width 50. The blood vessel constrictor 34 is made from a pliable material such as, but not limited to, nylon so that the blood vessel constrictor 34 can be wrapped around the leg 16(C) by aligning the longitudinal length 44 with the length of the leg 16 and wrapping the blood vessel constrictor 34 around the leg such that the ends 46 and 48 are aligned. The ends 46 and 48 are adapted to couple together by fasteners such as snaps, Velcro, etc.

FIG. 4 illustrates the blood vessel constrictor 34 as seen along line A-A of FIG. 3. The blood vessel constrictor 34 includes an inner wall 52 and an outer wall 54, which together define a chamber 56. In operable position, the blood vessel constrictor 34 is wrapped around the leg such that the inner wall 52 is adjacent to the leg.

The pressure line 38 extends from the outer wall 54 to the actuator 32 (FIG. 1). Responsive to fluid 58 being pumped into the chamber 56, the chamber 56 expands. In some embodiments, the outer wall 54 has an elasticity coefficient different from the inner wall 52 such that the outer wall 54 is stiffer so that expansion occurs primarily at the inner wall 52.

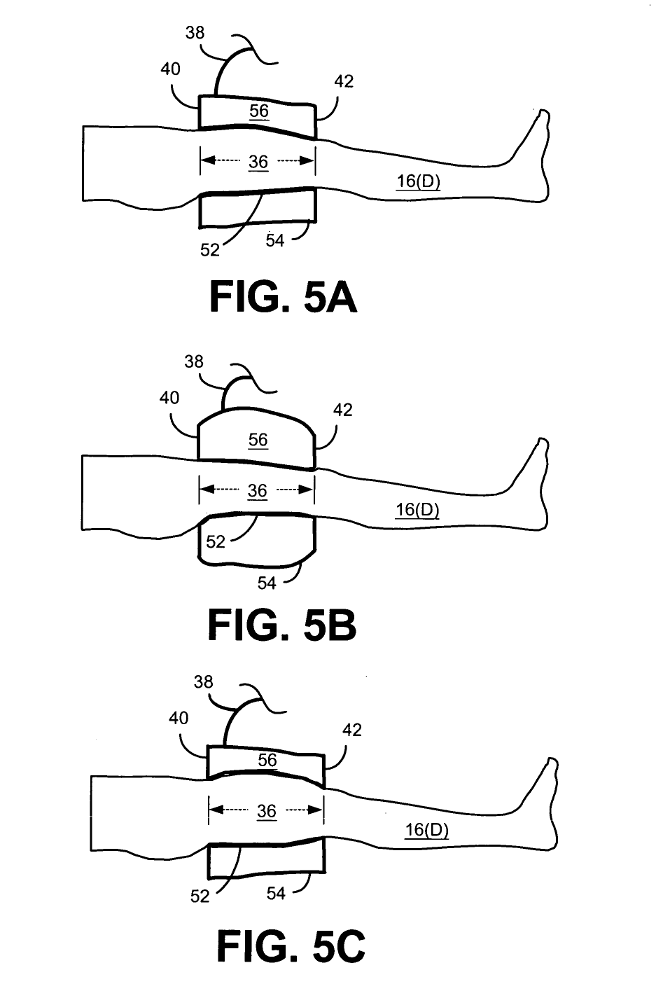

FIGS. 5A-5C illustrates cross-sectional views of the blood vessel constrictor 34 in operable position wrapped around leg 16(C). In FIG. 5A, the blood vessel constrictor 34 is in its relaxed state i.e.; pressure within the chamber 56 is approximately equal to atmospheric pressure. Consequently, leg 16(C) is also at atmospheric pressure, and blood pressure within the leg corresponds to the natural blood pressure of the patient 12.

In FIG. 5B, the blood vessel constrictor 34 is pressurized, i.e., the pressure is greater than the atmospheric pressure. Pressurization of the blood vessel constrictor 34 causes the inner wall 52 to constrict against the leg 16, which in turn causes blood flow out of the leg through the venial restrictor 28. The arterial restrictor 26 inhibits arterial reverse blood flow, i.e., and blood flow towards the heart 18 through the artery 20.

In FIG. 5C, the blood vessel constrictor 34 is in suction mode; the pressure within the chamber 52 is less than atmospheric pressure. Consequently, the pressure experienced by the therapeutic portion 36 of the leg 16 is less than the atmospheric pressure and, the leg expands accordingly. The expansion of the therapeutic portion 36 of the leg 16 causes blood to flow into the therapeutic portion 36 via artery 20. The venial blood restrictor 28 inhibits reverse venial blood flow, i.e., and blood flow through the vein 24 away from the heart 18.

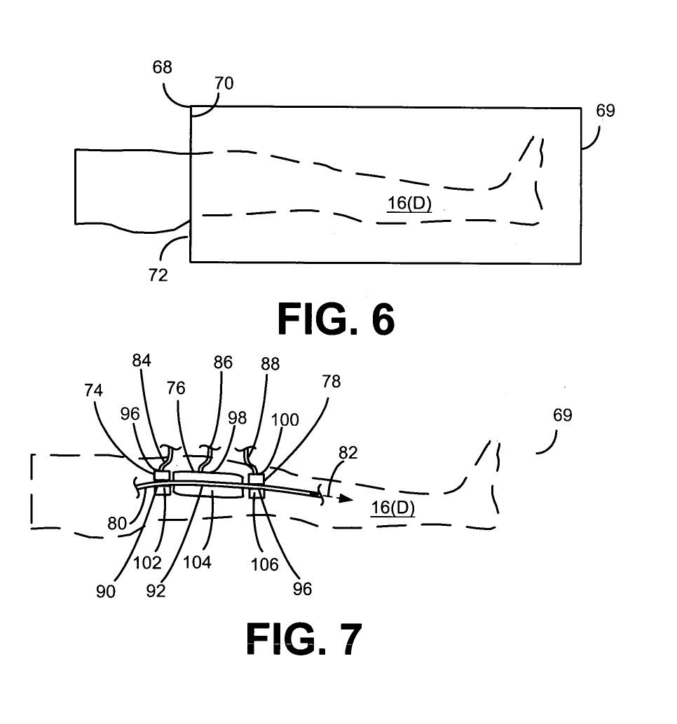

FIG. 6 illustrates another embodiment of a blood pumping system. The leg 16 (D) of the patient 12 extends into a pressure/vacuum chamber 66. The pressure/vacuum chamber 66 is generally cylindrical in shape having an open end 68, which defines an opening 70 for receiving the leg 16, and a closed end 69.

A curtain 72 is attached to the open end 68. The curtain 72 is adapted to fit around the torso 14 of the patient 12 such that a generally airtight seal is formed around the torso 14 of the patient. The pressure/vacuum chamber 66 is coupled to the actuator 32, via the pressure line 38 as described in FIG. 1. The controller 30 controls the pressure in the pressure/vacuum chamber 66, and the pressure is raised to compress/pump blood from the legs 16 into the torso 14 and lowered to draw blood from the torso 14 into the leg 16.

FIG. 7 illustrates components of an internal blood pumping system 73 in cross-sectional view. A first blood flow restrictor 74, a blood vessel constrictor 76 and a second blood flow restrictor 78 are serially disposed in the leg 16(D) of the patient 12 around a blood vessel 80, which can be a vein or an artery. Arrow 82 represents the natural blood flow direction within the blood vessel 80. Thus, blood vessel 80 is illustrated as an artery, but this is merely for the sake of illustration. The first blood flow restrictor 74, the blood vessel constrictor 76 and the second blood flow restrictor 78 are each connected to a pressure line 84, 86 and 88, respectively, and the pressure lines 84, 86 and 88 are connected to the actuator 32 (FIG. 1).

The first and second blood flow restrictors 74 and 78, respectively, and the blood vessel constrictor 76, each have an inner wall 90, 92, and 94, respectively, and an outer wall 96, 98 and 100, respectively, which define chambers 102, 104 and 106, respectively. The inner walls 92, 94 and 96 abut the blood vessel 80 and are made from a pliable/elastic material. Each one of the inner walls 92, 94 and 96 expands inward towards the blood vessel 80 in response to pressure in its chamber 102, 104 and 106, respectively. In some embodiments, the inner walls expand outward away from the blood vessel 80, in response to a partial vacuum, i.e., suction, in its chamber 102, 104 and 106, respectively.

Generally, the first and second blood flow restrictors 74 and 78 are smaller than the blood vessel constrictor 76 and function as valves to permit blood flow into and out of the region of the blood vessel 80 that is circumscribed by the blood vessel constrictor 76. The blood vessel constrictor 76 extends along and circumscribes a portion of the blood vessel 80. The blood vessel constrictor 76 is adapted to constrict the circumscribed portion of the blood vessel 80 so as to squeeze blood therefrom. In some embodiments, the portion of the blood vessel 80 that is circumscribed by the blood vessel constrictor 76 is enlarged from its natural state to increase the volume of blood within the circumscribed portion. Typically, the enlarged portion of the blood vessel 80 has a diameter that is larger than the natural diameter of the blood vessel. The increased diameter of the enlarged portion can be the result of grafting additional veins/arteries onto the blood vessel or causing the blood vessel grow such that its diameter increases.

In operation, the controller 30 signals the actuator 32 to cause the internal blood pump system 73 to pump blood through the blood vessel 80 in the natural blood flow direction 82 by sequentially and cyclically compressing and relaxing the first blood flow restrictor 74, the blood vessel constrictor 76, and the second blood flow restrictor 78. An exemplary cyclic sequence is shown in Table 1.

TABLE 1
Exemplary cycle of the internal blood pumping system.

| | First Blood | | Second Blood |
|---|---|---|---|
| Stroke | Restrictor | Pump | Restrictor |
| A | Constricted | Constricted | Open |
| B | Open | Constricted | Constricted |
| C | Open | Open | Constricted |
| D | Constricted | Open | Open |

In stroke A, the first blood restrictor 74 and the blood vessel constrictor 76 are constricted, i.e., pressurized such that they are applying pressure to the blood vessel 80. The second blood restrictor 78 is open, i.e., no inward pressure or minimal inward is being applied to the blood vessel 80. In some embodiments, components of the internal blood pumping system can be configured to apply a partial vacuum or suction to the blood vessel 80 when the components are in the “open” state, thereby causing the blood vessel 80 to expand. Stroke A corresponds to the state of the internal blood pumping system after blood has been pumped therefrom.

In stroke B, the first blood restrictor 74 opens, the second blood restrictor 78 constricts, while the blood vessel constrictor 76 remains constricted. The constriction of blood vessel constrictor 76 prevents or inhibits reverse blood flow.

In stroke C, while the first blood restrictor 74 remains open and the second blood restrictor 78 remains constricted, the blood vessel constrictor 76 opens, thereby allowing blood to flow into the blood vessel that is circumscribed by the blood vessel constrictor 76. The constricted second blood restrictor 78 prevents reverse blood flow.

In stroke D, while the blood vessel constrictor 76 remains open, the first blood restrictor 74 constricts and the second blood restrictor 78 opens. The constriction of the first blood restrictor 74 prevents or inhibits reverse blood flow when the blood vessel constrictor is constricted, and likewise, the opening of the second blood restrictor 78 enables/facilitates blood flow out of the internal blood pumping system 73 when the blood vessel constrictor 76 constricts.

It should be remembered that table 1 is merely illustrative of one possible cycle and that other cycles having more or fewer strokes are also possible. Thus, table 1 is a non-limiting example and other cycles are included within the scope of the invention.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

Therefore, having thus described the disclosure, at least the following is claimed:

1. A system for pumping blood through a body, the system comprising:
a blood restrictor that allows blood flow though a blood vessel in a first direction and inhibits blood flow in the opposite direction;
a blood vessel constrictor that in response to a change in pressure constricts to causes blood within the blood vessel to flow through said blood restrictor.

2. The system of claim 1, wherein the blood restrictor comprises a one-way check valve deposed in the vessel.

3. The system of claim 2, wherein the vessel is within the body.

4. The system of claim 2, wherein the blood vessel constrictor comprises an inflatable cuff disposed on a portion of the body, the cuff is adapted to inflate and deflate, wherein inflation of the cuff causes the cuff to constrict and apply pressure to body.

5. The system of claim 1, wherein blood vessel constrictor comprises a pressure chamber adapted to have at least a portion of the body disposed therein, the pressure chamber adapted to operate at a first pressure, a second pressure, and pressures therebetween.

6. The system of claim 5, wherein the second pressure is less than the ambient atmospheric pressure.

7. The system of claim 1, wherein the blood vessel constrictor comprises an inflatable cuff disposed in a portion of the body around the vessel.

8. The system of claim 7, wherein the blood restrictor comprises a second inflatable cuff disposed around the vessel, responsive to inflation, the inflated second cuff pinches a region of the vessel and inhibits blood flow therethrough.

9. A system for pumping blood through a body, the system comprising:
a first blood restrictor that allows blood to flow in a first direction though an artery and inhibits reverse blood flow in the artery;
a second blood restrictor that allows blood to flow in a second direction though a vein and inhibits reverse blood flow in the vein; and
a blood vessel constrictor that in response to a change in pressure causes blood within the body to flow such that blood is moved through the body.

10. The system of claim 9, wherein the first and second blood restrictors comprise one-way check valves.

11. The system of claim 9, wherein the first and second blood restrictors comprise animal heart valves.

12. The system of claim 9, wherein the blood vessel constrictor comprises an inflatable cuff disposed on a portion of the body, the cuff is adapted to inflate and deflate, wherein inflation of the cuff causes the cuff to apply pressure to the body.

13. The system of claim 9, wherein blood vessel constrictor comprises a pressure chamber adapted to have at least a portion of the body disposed therein, the pressure chamber adapted to operate at a first pressure, a second pressure, and pressures therebetween.

14. A system for pumping blood through a blood vessel of a body, the system comprising:
a first blood restrictor disposed about a portion of a blood vessel that allows blood to flow in a first direction though the blood vessel and inhibits reverse blood flow through the blood vessel,
a second blood restrictor disposed about a second portion of the blood vessel that allows blood to flow in the first direction though the blood vessel and inhibits reverse blood flow through the blood vessel; and
a blood vessel constrictor disposed around the blood vessel between said first and second blood restrictors responsive to the beat of the heart for urging blood along the blood vessel.

15. The system of claim 14, wherein the first and second blood restrictors comprise an inflatable cuff.

16. The system of claim 14, wherein the blood vessel constrictor comprises an inflatable cuff.

17. A method of assisting the heart of a human body to move blood through the blood vessels of the body, comprising:
alternately applying pressure to and relieving pressure from the exterior of a blood vessel of the body.

18. The method of claim 17, and further including restricting the flow of blood in one direction through the blood vessel.

19. The method of claim 17, and further including the step of timing the application of pressure to the exterior of a blood vessel with the heart beat of the body.
